(19) United States
(12) Patent Application Publication
(10) Pub. No.: US 2009/0075972 A1
(43) Pub. Date: Mar. 19, 2009

(54) Use of Midostaurin for Treating Gastrointestinal Stromal Tumors

(76) Inventor: Jan Cools, Leuven (BE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/574,342

(22) PCT Filed: Aug. 30, 2005
(86) PCT No.: PCT/EP05/09337

§ 371 Date: Oct. 16, 2008

Related U.S. Application Data
(60) Provisional application No. 60/605,771, filed on Aug. 31, 2004.

(51) Int. Cl. A61K 31/553; A61P 35/00

(52) U.S. Cl. 514/211.08

(57) ABSTRACT

The present invention relates to the use of midostaurin, in free form or in pharmaceutically acceptable salt form in the manufacture of a pharmaceutical composition for the treatment of gastrointestinal stromal tumors, and to a method of treatment of warm-blooded animals, preferably humans, in which a therapeutically effective dose of midostaurin is administered to an animal suffering from said disease or condition.

The present invention relates to the use of midostaurin, in free form or in pharmaceutically acceptable salt form in the manufacture of a pharmaceutical composition for the treatment of gastrointestinal stromal tumors, e.g. gastrointestinal tumors resistant to Compound I, and to a method of treatment of warm-blooded animals, preferably humans, in which a therapeutically effective dose of midostaurin animal suffering from said disease or condition mentioned above.

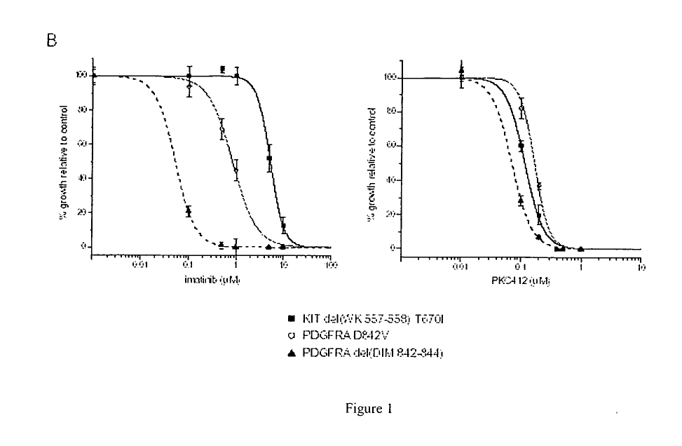

### Description of FIG. 1.

Panel B: dose response curves of imatinib or PKC412 for Ba/F3 cells expressing KIT ΔWK557-558/T670I, PDGFRA D842V or ΔDIM842-844 mutations.

Gastrointestinal stromal tumours are a recently characterized family of mesenchymal neoplasms, which originate from the gastrointestinal tract, 60 to 70% of all GISTs originate from the stomach. In the past, these tumours were variously classified as leiomyoma, leiomyoblastoma, or leiomyosarcoma. However, it is now clear that GISTs represent a distinct clinicopathologic set of diseases based on their unique molecular pathogenesis and clinical features.

GIST is a relatively rare condition and has an estimated incidence of about 20 cases/million, GIST is the most common mesenchymal neoplasm of the gastrointestinal tract. Until recently the only available therapy has been surgical resection. The limited value of conventional cytotoxic chemotherapy and radiation therapy has resulted in advanced GIST being an invariably progressive and fatal condition, the median survival of patients varying from 20 months, e.g. metastatic GIST, to a year or less, e.g. post-surgical recurrence.

The most likely causative oncogenic molecular event in the vast majority of GISTs is an activating mutation of KIT or platelet-derived growth factor receptor A, abbreviated as PDGFRA. As a result signaling pathways are activated that promote cell proliferation and/or survival. Imatinib mesylate specifically inhibits the receptor tyrosine kinases PDGFRs, KIT, ABL, and ARG, and induces high response rates in patients with GISTs. To date, imatinib therapy remains the only effective, systemic treatment for this disease. Clinical and experimental observations linked the response to the presence and the type of KIT/PDGFRA mutations in the tumor, with those carrying KIT exon 11 mutations being the most sensitive to treatment. KIT-D816V and PDGFRA-D842V mutations, affecting the kinase catalytic domain, interfere with the binding of imatinib and render the drug primary ineffective. The majority of GIST patients develop resistance during therapy, after differing degrees of initial response to the drug. The investigation of other malignancies treated with imatinib, such as chronic myeloid leukemia (CML), or chronic eosinophilic leukemia (CEL), indicates that resistance to this inhibitor can be caused by distinct molecular mechanisms. The majority of CML patients with imatinib-resistance have a clonal expansion of leukemic cells harboring novel mutant BCR-ABL alleles or expressing higher levels of the fusion protein due to BCR-ABL amplification. The development of resistance to imatinib in CEL can be associated with a secondary mutation within catalytic domain of FIPL1-PDGFRA fusion protein. Preliminary studies in GIST patients with imatinib-resistant progressive stage of disease indicated that in a majority of tumors KIT activation still continued to play a functional role, with acquired mutations of KIT kinase domain or genomic amplification of KIT gene as a causative factors in a subset of patients.

Imatinib is a small molecule selectively inhibiting specific tyrosine kinases that has emerged recently as a valuable treatment for patients with advanced GIST. The use of imatinib as monotherapy for the treatment of GIST has been described in PCT publication WO 02/34727, which is here incorporated by reference. However, it has been reported that primary resistance to imatinib is present in a population of patients, for example 13.7% of patients in one study. In addition, a number of patients acquire resistance to treatment with imatinib. More generally this resistance is partial with progression in some lesions, but continuing disease control in other lesions. Hence, these patients remain on imatinib treatment but with a clear need for additional or alternative therapy.

Imatinib is 4-(4-methylpiperazin-1-ylmethyl)-N-[4-methyl-3-(4-pyridin-3-yl)pyrimidin-2-ylamino)phenyl]-benzamide having the formula I

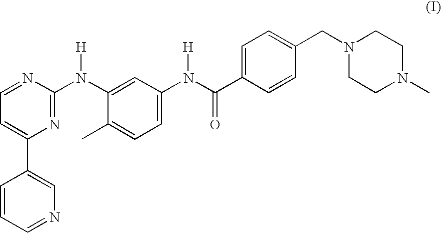

The preparation of imatinib and the use thereof, especially as an anti-tumour agent, are described in Example 21 of European patent application EP-A-0 564 409, which was published on 6 Oct. 1993, and in equivalent applications and patents in numerous other countries, e.g. in U.S. Pat. No. 5,521,184 and in Japanese patent 2706682, all of which are incorporated by reference herein.

It has now surprisingly been found that midostaurin, a protein kinase C inhibitor, possesses therapeutic properties which render it useful for the treatment of gastrointestinal stromal tumors, e.g. for the treatment of imatinib-resistant gastrointestinal stromal tumors.

Protein kinase C, herein after abbreviated as PKC, is one of the key enzymes in cellular signal transduction pathways, and it has a pivotal role in the control of cell proliferation and differentiation. PKC is a family of serine/threonine kinases. At least 12 isoforms of PKC have been identified, and they are commonly divided into three groups based on their structure and substrate requirements. PKC expression has been found to be elevated in human breast tumor biopsies as compared with normal breast tissues, and high PKC expression has been considered as a biological marker for malignancy in human astrocytomas. One of the PKC isoforms, PKCθ, is a positive regulator of survival signaling in T cells. Interestingly, PKCθ is constitutively phosphorylated in GIST. Thus, PKCθ may be considered a potential target kinase for therapeutic interventions in GIST. In particular, PKC inhibitors are beneficial in the treatment of imatinib resistant GISTs.

Accordingly, the present invention relates to a method of treating GIST, which comprises administering midostaurin, to a patient with GIST, e.g. with imatinib-resistant GIST.

Midostaurin according to the invention is N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide of the formula (II):

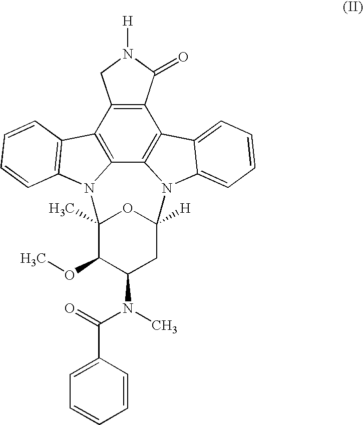

or a salt thereof, hereinafter: “Compound of formula II or midostaurin”.

Compound of formula II or midostaurin [International Nonproprietary Name] is also known as PKC412.

Midostaurin is a derivative of the naturally occurring alkaloid staurosporine, and has been specifically described in the European patent No. 0 296 110 published on Dec. 21, 1988, as well as in U.S. Pat. No. 5,093,330 published on Mar. 3, 1992, and Japanese Patent No. 2 708 047. Midostaurin described in these documents are incorporated into the present application by reference. Midostaurin and its manufacturing process has been specifically described in many documents, well known by the man skilled in the art.

In each case where citations of patent applications or scientific publications are given in particular for midostaurin, the subject-matter of the final products, the pharmaceutical preparations and the claims are hereby incorporated into the present application by reference to these publications.

The term “imatinib-resistant or imatinib-resistance” as used herein defines a lack, a reduction or a loss of therapeutic effectiveness of imatinib in the treatment of gastrointestinal stromal tumors.

The invention relates to the use of midostaurin, also known as PKC412, or a pharmaceutically acceptable salt thereof, for the manufacture of a medicament for the treatment of gastrointestinal stromal tumours, herein after abbreviated as GIST, e.g. imatinib-resistant GIST, and to a method of treating warm-blooded animals, including humans, suffering from GIST by administering to a said animal in need of such treatment an effective amount of midostaurin, or a pharmaceutically acceptable salt thereof.

The present invention relates to a method of treating GIST, e.g. with imatinib-resistant GIST, which comprises administering midostaurin, to a patient with GIST, e.g. with imatinib-resistant GIST.

The precise dosage of midostaurin to be employed for treating the diseases and conditions mentioned hereinbefore depends upon several factors including the host, the nature and the severity of the condition being treated, the mode of administration. In general, satisfactory results are achieved when midostaurin is administered parenterally, e.g., intraperitoneally, intravenously, intramuscularly, subcutaneously, intratumorally, or rectally, or enterally, e.g., orally, preferably intravenously or, preferably orally, intravenously at a daily dosage of 0.1 to 10 mg/kg body weight, preferably 1 to 5 mg/kg body weight. In human trials a total dose of 225 mg/day was most presumably the Maximum Tolerated Dose (MTD). A preferred intravenous daily dosage is 0.1 to 10 mg/kg body weight or, for most larger primates, a daily dosage of 200-300 mg. A typical intravenous dosage is 3 to 5 mg/kg, three to five times a week.

Midostaurin is administered orally in dosages up to about 300 mg/day, for example 100 to 300 mg/day. The midostaurin is administered as a single dose or split into two or three doses daily, preferably two doses. A particularly important dose is 200-225 mg/day, in particular 100 mg twice a day (200 mg/day total). The upper limit of dosage is that imposed by side effects and can be determined by trial for the patient being treated.

The instant invention also concerns a method wherein the therapeutically effective amount of midostaurin is administered to a mammal subject 7 to 4 times a week or about 100% to about 50% of the days in the time period, for a period of from one to six weeks, followed by a period of one to three weeks, wherein the agent is not administered and this cycle being repeated for from 1 to several cycles.

Usually, a small dose is administered initially and the dosage is gradually increased until the optimal dosage for the host under treatment is determined. The upper limit of dosage is that imposed by side effects and can be determined by trial for the host being treated.

Midostaurin may be combined with one or more pharmaceutically acceptable carriers and, optionally, one or more other conventional pharmaceutical adjuvants and administered enterally, e.g. orally, in the form of tablets, capsules, caplets, etc. or parenterally, e.g., intraperitoneally or intravenously, in the form of sterile injectable solutions or suspensions. The enteral and parenteral compositions may be prepared by conventional means.

The infusion solutions according to the present invention are preferably sterile. This may be readily accomplished, e.g. by filtration through sterile filtration membranes. Aseptic formation of any composition in liquid form, the aseptic filling of vials and/or combining a pharmaceutical composition of the present invention with a suitable diluent under aseptic conditions are well known to the skilled addressee.

Midostaurin may be formulated into enteral and parenteral pharmaceutical compositions containing an amount of the active substance that is effective for treating the diseases and conditions named hereinbefore, such compositions in unit dosage form and such compositions comprising a pharmaceutically acceptable carrier.

Examples of useful compositions are described in the European patents No. 0 296 110, No. 0 657 164, No. 0 296 110, No. 0 733 372, No. 0 711 556, No. 0 711 557.

The preferred compositions are described in the European patent No. 0 657 164 published on Jun. 14, 1995. The described pharmaceutical compositions comprise a solution or dispersion of midostaurin in a saturated polyalkylene glycol glyceride, in which the glycol glyceride is a mixture of glyceryl and polyethylene glycol esters of one or more C₈-C₁₈ saturated fatty acids.

The present invention relates to the use of midostaurin, or a pharmaceutically acceptable salt thereof for the preparation of a medicament for the treatment of GIST, e.g. imatinib-resistant GIST, with the proviso that midostaurin is not administered together, sequentially, or separately with imatinib.

The present invention relates to the use of midostaurin or a pharmaceutically acceptable salt thereof for the treatment of GIST, e.g. imatinib-resistant GIST, wherein imatinib is not used for the treatment of said GIST, e.g. imatinib-resistant GIST.

The present invention relates to the use of midostaurin or a pharmaceutically acceptable salt thereof wherein midostaurin is used as an anti-tumor agent for the treatment of GIST, e.g. imatinib-resistant GIST.

The present invention further relates to packaged midostaurin what includes instructions to use midostaurin, or salts thereof, together for the treatment of GIST, e.g. imatinib-resistant GIST.

In one aspect the present invention provides a method of treating GIST comprising administering midostaurin in an amount which is therapeutically effective against GIST to a warm-blooded animal, particularly a human, in need thereof. More particularly, the present invention provides a method of treating a patient suffering from GIST, which comprises administering an effective amount of midostaurin, or a pharmaceutically acceptable salt thereof, to the patient. More particularly, the present invention provides a method of treating a patient suffering from GIST, which comprises administering an effective midostaurin, or a pharmaceutically acceptable salt thereof, to the patient, wherein the midostaurin is administered in a dose of 100 to 300 mg daily, particularly 150 to 250 mg daily, most particularly 200 mg daily, as an oral pharmaceutical preparation

### EXAMPLE 1

### Midostaurin Pharmaceutical Preparations

### Composition A:

Gelucire 44/14 (82 parts) is melted by heating to 60° C. Powdered Midostaurin (18 parts) is added to the molten material. The resulting mixture is homogenised and the dispersion obtained is introduced into hard gelatin capsules of different size, so that some contain a 25 mg dosage and others a 75 mg dosage of the Midostaurin. The resulting capsules are suitable for oral administration.

### Composition B:

Gelucire 44/14 (86 parts) is melted by heating to 60° C. Powdered Midostaurin (14 parts) is added to the molten material. The mixture is homogenised and the dispersion obtained is introduced into hard gelatin capsules of different size, so that some contain a 25 mg dosage and others a 75 mg dosage of the Midostaurin. The resulting capsules are suitable for oral administration.

Gelucire 44/14 available commercially from Gattefossé; is a mixture of esters of C8-C18 saturated fatty acids with glycerol and a polyethylene glycol having a molecular weight of about 1500, the specifications for the composition of the fatty acid component being, by weight, 4-10% caprylic acid, 3-9% capric acid, 40-50% lauric acid, 14-24% myristic acid, 4-14% palmitic acid and 5-15% stearic acid.

A preferred example of Gelucire formulation consists of:

### Gelucire (44/14): 47 g

Midostaurin: 3.0 g filled into a 60 mL Twist off flask

### Composition C: an Example of Soft Gel Will Contain the Following Microemulsion:

| | | |
|---|---|---|
| Cornoil glycerides | 85.0 | mg |
| Polyethylenglykol 400 | 128.25 | mg |
| Cremophor RH 40 | 213.75 | mg |
| Midostaurin | 25.0 | mg |
| DL alpha Tocopherol | 0.5 | mg |
| Ethanol absolute | 33.9 | mg |
| Total | 486.4 | mg |

### EXAMPLE 2

PKC412 interacts strongly with ATP binding sites of the conventional PKCs, FLT3, PDGFRs, VEGFRs, KIT and the CDK1-cyclin B complex. Notably, PKC412 was shown to exhibit full inhibitory activity against the imatinib-resistant T674 I mutant form of FIPL1-PDGFRA in refractory CEL patients, see e.g. Cools J., et al., Cancer Cell 2003; 3:459-469. The catalytic sites of tyrosine kinases are highly conserved, and the T674I mutation in PDGFRA corresponds to the T315I mutation in ABL and the T670I mutation in KIT, the resistant mutations in progressive BCR-ABL positive CML and in KIT mutant GISTs patients, respectively. The mechanisms of resistance to imatinib in 26 patients with GISTs refractory to imatinib is investigated and the use of PKC412 to overcome the clinical resistance to imatinib in those patients due to the recurrent KIT-T670I or -V654A, and PDGFRA-D842V kinase domain mutations is explored.

### Materials and Methods

Patients: Progressive tumors from 26 patients treated with imatinib in the Department of Oncology, University Hospital Leuven were evaluated. There are 20 men and 6 women, with a median age of 53 years (range, 37 to 77 years). Twenty-two out of 26 patients had the primary tumor surgically removed. Chemotherapy and/or radiotherapy was applied in the advanced stage of the disease in 13 patients, prior to imatinib treatment. Patients whose tumor progressed but who were otherwise in good clinical condition were eligible to dose increase Up to 1000 mg daily. Dose escalation decisions were based on data from patients treated at least 4 weeks. Lesions were reassessed after one month, three months, and every six months thereafter. Progression was based on clinical examination and CT/PET imaging, and defined according to criteria previously published, see e.g. Van Oosterom AT et al., Lancet 2001; 358:1421-1423. Histopatlhological and molecular changes during the treatment are evaluated in selected consenting patients by means of serial tumor biopsies.

Pathology: Histopathologic and immunohistochemical analyses are performed on tissue embedded in paraffin. Polyclonal antibodies against CD117 (A4502, dilution 1/250, DAKO, Denmark) and avidin-biotin-peroxidase complexes are used without any antigen retrieval.

Fluorescence in situ hybidization (FISH). Dual-color interphase FISH analysis is performed on 4 μm paraffin embedded tissue sections of tumor biopsies obtained before imatinib treatment (18 cases), or on touch preparations from fresh biopsies of imatinib-resistant lesions (all 26 cases). Digoxigenin- or biotin labeled BAC clones for KIT/4q12 (RP11-568A2) or PDGFRA/4q12 (RP11-24O11) are co-hybridized with SpectrumGreen- or SpectrumOrange-labeled chromosome 4 centromeric probes (CEP4, Vysis Inc., Downers Grove, Ill., USA), respectively, as previously described. 21 The FISH data are collected on a Leica DMRB (Leica, Wetzlar, Germany) fluorescence microscope equipped with a cooled black and white charged couple device camera (Photometrics, Tuscon, Ariz.), run by Quips SmartCapture™ FISH Imaging Software (Vysis, Bergisch-Gladbach, Germany). eHundred interphase nuclei are evaluated, and the ratio of KIT/PDGFRA to CEP4 was calculated. A ratio of ≧2 is defined as specific KIT/PDGFRA amplification.

Sequence analysis: Genomic DNA is extracted from snap-frozen tissue using the High Pure PCR Template Preparation Kit (Roche, Mannheim, Germany) Exons 9, 11, 13, 14, 15 and 17 of the KIT, and exons 12 and 18 of the PDGFRA are amplified by the polymerase chain reaction (PCR) as previously described, see e.g. Debiec-Rychter M et al., J Pathol 2004; 202:430-438. The PCR products were purified (Microcon PCR, Millipore, Mass., USA) and screened for mutations by denaturing high-performance liquid chromatography on a Transgenomic WAVE DHPLC system (DIIPLC; Transgenomic, Inc., UK). Samples showing an aberrant elution profile were re-amplified and sequenced.

Western-blot: Snap-frozen tumor specimens sufficient for preparation of cell lysates were available from ten refractory GISTs. Cell lysis, SDS-PAGE and immunoblotting were carried out as described.²¹ Membranes (Amnersham Pharmacia Biotechnology, UK) were immunoblotted overnight using anti-phospho-KIT (Y703) (Zymed, San Francisco, Calif.) antibody at dilution of 1:500. The HRP-conjugated anti-rabbit IgG was used at a dilution of 1:2500 and visualized with Enhanced Chemiluminescence (Pierce). Membranes were then stripped and re-blotted to determine total protein levels using an antibody recognizing total KIT protein (anti-CD117, A4502, DAKO, Glostrup, Denmark).

Primary resistant GIST cells response assay: Imatinib mesylate and PKC412, the crystalline compounds are dissolved at 10 mM in 100% DMSO (Sigma) and aliquots are kept at −80° C. Experiments are performed with serial dilutions of the 10 mM stock. Controls are performed with solvent (DMSO) dilutions. Primary cells are obtained from collagenase disaggregated progressive tumor specimens, seeded at 60-70% confluence in 100-mm cell culture dishes (Corning Inc., Corning, N.Y.) and grown for three days in DMEM supplemented with 10% fetal bovine serum, 0.1 mM nonessential amino acids, and 1.0 mM sodium pyruvate. Cells are exposed to either imatinib mesylate, PKC412 or vehicle alone for 90 min, washed with 10 ml of cold PBS, and lysed in buffer [1% NP40, 50 mM Tris-HCl pH 8.0, 150 mM NaCl, supplemented with complete protease inhibitor cocktail tablets (Boehringer Mannheim GmbH, Mannheim, Germany) and 0.2 mM sodium orthovanadate (Sigma, St. Louis, Mo.)].

Construct: Mutant PDGFRA and KIT cDNA are obtained by RT-PCR on RNA isolated from progressive tumors. The cDNA's are cloned into the retroviral vector pMSCV-puro (Clontech).

Cell culture: 293T cells are grown in DMEM supplemented with 10% FCS. Ba/F3 cells are grown in RPMI-1640 supplemented with 10% FCS and interleukin-3 (1 ng/ml). Virus as produced as described previously, see e.g. Cools J. et al., N Engl J Med 2003; 348:1201-1214.

.Ba/F3 cells transduced with the different constructs are selected with puromycin (2 μg/ml). To test for factor independent growth, Ba/F3 cells are washed 3 times in PBS and new cultures are initiated in the absence of interleukin-3. Cells that became independent on interleukin-3, are maintained in the absence of interleukin-3. For dose-response curves, Ba/F3 cells are grown in 24-well plates with different concentrations of inhibitor. The number of viable cells is determined at the start and after 24 hrs, using the AqueousOne solution (Promega).

Results: Progressive tumors from 26 patients treated with imatinib are evaluated. The median time from the diagnosis to the proven malignancy of the disease is 48 weeks (range, 0 to 265 weeks), while the median time from the diagnosis to imatinib treatment is 91 weeks (range, 6 to 304 weeks). Fifteen patients (57.6%) achieved partial remission, and 10 patients (38.4%) showed stable disease during imatinib treatment, with an average duration of event free survival of 48 weeks (range 16 to 200 weeks).

Histopathology: Twenty-five primary GISTs reveal spindle cell and one had mixed morphology. CD117 antigen expression is demonstrated in each primary tumor and in 24 out of 26 (92%) progressive biopsies. Two imatinib-resistant GISTs invert their histologic appearance from spindle to epithelioid type and their immunophenotype, becoming CD117 negative (data not shown).

Mutation analysis: A combination of D-HPLC and direct sequencing revealed KIT mutations in 25 out of 26 (96.1%) base-line GIST biopsies, see Table 1. Nineteen tumors harbored exon 11 juxtamembrane mutations and six carried exon 9 mutations. None pre-treatment tumor specimen had mutations in PDGFRA or more than one mutation in KIT. One tumor had no identifiable KIT or PDGFRA sequence alteration in the examined exons. While no point mutations of the KIT kinase domain are detected in the tumors before imatinib treatment, six distinct secondary KIT mutations are identified in 12/26 (48%) patients at the time of progression, after a median of 77 weeks (range 16-188) on therapy. Four patients had a V654A and three patients had a T670I substitution, while the remaining patients carried D716N, D816G, D820Y, D820E or N822K mutations. One patient with an original KIT G565R mutation acquired a D842V point mutation in PDGFRA, not detectable in the primary tumor from this patient.

FISH analysis. FISH analyses reveal amplification of KIT in 2 of 26 (7.7%) progressive tumors. In the primary non-responding tumor from patient 26, KIT amplification is associated with simultaneous amplification of PDGFRA (data not shown). No KIT or PDGFRA mutations are found in the tumor from this patient, neither before treatment nor during progression of the disease. In one patient, KIT amplification (up to 5-fold) is not associated with increased PDGFRA copy number. This case harbored a primary KIT mutation, but secondary mutations are not identified during progression. In six imatinib-resistant specimens, loss of KIT/PDGFRA/CEP4 loci is revealed by interphase FISH analysis. While in three of the tumors, this hemizygosity is already observed in the base-line tumor biopsies, in three other specimens, it is only present in the progressive lesions. Within the latter, however, marked heterogeneity in the number of KIT/CEP4 signals per nuclei is encountered (range from 0 to 4). Particularly, 23% of cells in progressive tumor biopsies from one patient showed bi-allelic loss of KIT/PDGFRA/CEP4.

KIT activation in resistant GISTs. KIT activation in 10 imatinib-resistant GISTs is evaluated by Western blotting with antibodies to KIT phosphotyrosine Y703 and total KIT. Eight specimens demonstrate KIT expression and various levels of constitutive KIT autophosphorylation. Four of these eight tumors have secondary KIT mutations, and for the remaining four the reason for the re-activation of KIT in imatinib-resistant tumor cells is unknown. Two resistant metastatic tumors totally lacked KIT expression, which is in line with the loss of CD117-positivity by immunohistochemistry, and the observed bi-alleic loss of KIT loci in one case.

Ex-vivo espouse of resistant GISTs to imatinib and PKC412: The effect of imatinib and PKC412 on the autophosphorylation of the KIT Y703 residue in cultured imatinib-resistant cells that harbored KITΔ557-558/T670I or KITInsAY502-503/V654A mutant isoforms is determined by Western blot. The results are compared with GIST882 cells, which carry a hemizygous KIT K642E mutation. Observations are standardized for total KIT expression using anti-KIT antibody. KIT protein is expressed and phosphorylated to a significant level in both resistant KITΔ557-558/T670I and KITIns503AY/V654A tumors and their in vitro cultured cell counterparts. The autophosphorylation of KIT is not affected by exposure of either primary cell line to imatinib (up to 5 μM). In contrast, 0.5 μM PKC412 reduced and 1 μM PKC412 totally inhibit KIT autophosphorylation of the mutant KITΔ557-558/T670I cells. Similarly, KIT autophosphorylation of the mutant KITIns503AY/V654A is reduced by PKC412 already at concentration 0.5 μM and completely inhibited at a ten-fold higher concentration of the drug.

Effect of imatinib and PKC412 on KIT and PDGFRA mutants in vitro: Mutant forms of KIT Δ557-558/T670I, and PDGFRA ΔDIM842-844 and D842V are expressed in Ba/F3 murine cells. Ba/F3 cells are IL3 dependent for their growth, but become IL3 independent upon the expression of many activated kinases, such as FIP1 L1-PDGFRA and BCR-ABL. Mutant KIT and PDGFRA proteins introduced in the Ba/F3 cells also confer factor independent growth, and are constitutively phosphorylated, confirming that these are activated kinases (data not shown). Dose response curves and analysis of the phosphorylation state of KITΔ557-558/T670I with imatinib confirmed the resistance to imatinib, with phosphorylation not completely inhibited at 10 μM imatinib (cellular IC₅₀˜5 μM). The PDGFRA D824V mutant also show resistance to imatinib, although to a lesser extent (cellular IC₅₀˜1 μM). The PDGFRA ΔDIM842-844 mutant serve as a control in this experiment. All 3 mutants are inhibited by PKC412 at concentrations below 1 μM, with PDGFRA D842V having the highest cellular IC₅₀ value of ˜200 nM (FIG. 1).

Preliminary studies described two categories of imatinib resistance: KIT-dependent or KIT-independent Mechanisms. ¹⁵ Based on our results, we conclude that re-activation of KIT is the most important mechanism for resistance. KIT is found to be phosphorylated (activated) in 8 of 10 progressive tumors that could be analyzed by Western blot during imatinib treatment. In 50% of these cases, reactivation of KIT is the consequence of secondary resistance mutations, while in the other 50% the cause for reactivation remains unknown. Sequencing KIT in its entirety in these samples may identify novel mutations in unexpected regions of KIT that render the protein insensitive to imatinib treatment. Alternatively, factors influencing intracellular drug delivery or clearance could result in inadequate receptor inhibition, with a consequent progression of the disease.

In the 26 patients in our study, acquired secondary KIT mutations are the most frequent event (48% of the cases) explaining resistance to imatinib. Six distinct secondary KIT mutations are identified in progressive tumors. All are single amino acid substitutions and all are present in addition to the activating KIT mutations identified in the base-line, non-treated tumors. To our knowledge, two recurrent KIT mutations, V654A and T670I, and three others, D716N, D820E and D816G, present in single cases, have not been previously reported in primary GISTs. This supports the close association of these mutations with the development of resistance to the drug. The D820Y and N822K mutations are previously described in imatinib non-treated GISTs. The activation loop mutations, e.g. D816G, D820E/Y, N822K, are likely to be activating mutations in KIT that also directly confer resistance to imatinib. The KIT D816V mutation in patients with systemic mastocytosis and in a subset of seminomas is associated with primary resistance to imatinib.

One tumor with a primary KIT G565R mutation acquires resistance to imatinib through a secondary PDGFRA D842V mutation. The D842V mutation is the most common activating PDGFRA mutation in GISTs, and is also proven to be imatinib-resistant. This mutation is an activating mutation that shows decreased sensitivity to imatinib. The observation that resistance to imatinib can occur through mutation of a different kinase, e.g. PDGFRA, identifies a previously not described mechanism of resistance. In general, resistance of a tumor dependent on an activated kinase sensitive to a small molecule inhibitor could occur by an activating mutation in a different kinase that is not sensitive to this inhibitor. It remains to be determined if this mechanism of resistance operates more frequently in GISTs and other tumors and leukemias, and whether it is the cause of resistance in the cases of our study in which we are unable to identify secondary genomic changes in KIT.

In two cases of this study, imatinib-resistance is associated with amplification of KIT or KIT/PDGFRA genes. In the latter, the patient showed primary resistance to imatinib with the massive progressive tumor growth, and consequently died five weeks from the start of imatinib administration. As the malignant stage of the disease in this patient lasted over one year and the patient was pretreated with high dose chemo- and radiotherapy before treatment with imatinib, the amplification was most likely already present in tumor cells before imatinib administration and further selected for in the presence of the drug. This finding indicates that KIT amplification may cause primary resistance, and cautions the use of classical chemotherapy in GISTs patients, which may add to the evolution of the clonal diversity associated with disease progression, with possible generation of the genetic changes influencing the response to the drug.

Two progressive tumors completely lost KIT expression, indicating KIT-independent mechanism of resistance. Interphase FISH analysis revealed selective growth of cells with the bi-allelic loss of targeted KIT/PDGFRA genes in one of these tumors, further underlining the escape from the receptor dependence. The shift to KIT/PDGFRA hemizygosity is observed in two tumors at the time of resistance to imatinib, which is associated with the appearance of secondary KIT mutations. Whether hemizygosity/homozygosity adds to insensitivity of recurrent mutants to imatinib is unclear and warrants further study.

In an attempt to define the imatinib sensitivity of the common KIT V654A and T670I mutations present in tumor cells at the time of progression, the inhibitory effect of imatinib on the ligand-independent KIT phosphorylation in cells harboring these mutations is examed using ex vivo assay. In both cases, KIT autophosphorylation is not inhibited at concentrations of imatinib as high as 5 μM, which is about the maximum level of imatinib that can be achieved in vivo. PKC412, an alternative KIT and PDGFR inhibitor, exerted inhibitory effect on both mutants at the concentrations that justify therapeutical use of the drug. The differential sensitivity to imatinib and PKC412 on KIT T670I mutant is further validated in vitro using transformed Ba/F3 murine cells. To further explore the sensitivity of other imatinib-resistant mutations to PKC412, Ba/F3 cells transfected with imatinib-resistant PDGFRA D842V mutant are tested. PKC412 efficiently inhibits the PDGFRA D842V mutant at the concentration of 1 μM, additionally emphasizing the in vitro potency of the drug for inhibition of tumors harboring different imatinib-resistant mutant isoforms. The existence of KIT-dependent and independent mechanisms of imatinib-resistance in GISTs patients is confirmed and reveals novel imatinib-resistant KIT mutant isoforms. It points to the acquisition of imatinib-resistant PDGFRA mutations as a cause of secondary resistance in a KIT positive tumor, and indicates the KIT amplification as the possible explanation not only for a secondary but also for a primary resistance to the drug. The sensitivity of KIT T670I and V654A, and PDGFRA D6842V mutations to PKC421 is evidenced. Given that individual kinase domain mutations exhibit differential sensitivity to alternative kinase inhibitors, it is crucial to tailor second-line therapy precisely to the underlying mechanism of resistance.

TABLE 1
KIT and PDGFRA tumor genotype 26 GISTs patients.

| | Genotype | |
|---|---|---|
| | Base-line biopsy | Secondary mutations ^{a} |
| Case | KIT | KIT or PDGFRA |
| 1 | PM K558N | |
| 2 | Del WK557-558 | |
| 3 | Del WK557-558 ^{d} | |
| 4 | Del WK557-558 ^{d} | |
| 5 | Del KVVE558-561 | |
| 6 | Del KVVEEI 558-563 | |
| 7 | Del VYIDPTQL 569-576 | |
| 8 | Del GNNYVYIDPTQLPYD565-579V | |
| 9 | PM V559G | KIT V654A (GTG→GCG) |
| 10 | PM L576P ^{d} | KIT V654A (GTG→GCG) ^{d} |
| 11 | Ins 574PT | KIT V654A (GTG→GCG) |
| 12 | Del WK557-558 | KIT D716N (GAT→AAT) |
| 13 | Del WK557-558 | KIT T670I (ACA→ATA) |
| 14 | Del WK557-558 | KIT T670I (ACA→ATA) ^{d} |
| 15 | Del KPMYEVQWK 550-558Q | KIT T670I (ACA→ATA) |
| 16 | Del VEEINGNNYVYIDPTQL560-576 | KIT D820E (GAT→GAA) |
| 17 | Del VYIDPTQL 569-576 | KIT D820Y (GAT→TAT) |
| 18 | Del VYIDPTQL 569-576 | KIT N822K (AAT→AAA) ^{d} |
| 19 | Ins 503AY | KIT V654A (GTG→GCG) |
| 20 | Ins 503AY | KIT D816G (GAC→GGC) |
| 21 | Ins 503AY | |
| 22 | Ins 503AY | |
| 23 | Ins 503AY | |
| 24 | Ins 503AY | |
| 25 | PM G565R | PDGFRA D842V (GAC→GTC) |
| 26 | WT | |

Abbreviations: WT—wild type;
^{a} mutations detected on the top of base-line mutant isoform;
^{b} range of KIT signals per nucleus;
^{d} hemizygous by sequencing

1. A method of treating a patient suffering from gastrointestinal stromal tumors, which comprises administering an effective amount of midostaurin of formula, 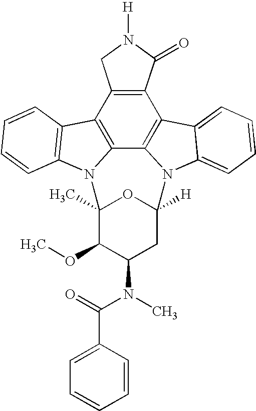
or a pharmaceutically acceptable salt thereof, to the patient in need thereof.

2. The method of claim 1 wherein the gastrointestinal stromal tumor is imatinib-resistant gastrointestinal stromal tumor.

3. The method of claim 2 wherein the midostaurin is administered in a dose of 100 to 300 mg daily.

4. The method of claim 3 wherein the dose is 150 to 250 mg daily.

5. The method of claim 4 wherein the dose is 200 mg daily.

6. The method of claim 1 wherein midostaurin is administered to the patient with the proviso that midostaurin is not to be used for simultaneous, separate or sequential use with imatinib.

7. Use of midostaurin for the preparation of a medicament for the treatment of gastrointestinal stromal tumors.

8. The use according to claim 7 wherein the gastrointestinal stromal tumors are resistant to therapy with imatinib.

9. The use of claim 8 wherein the midostaurin is to be administered in a dose from 150 to 250 mg daily.

10. The use of claim 9 wherein the dose to be administered is 200 mg daily.

11. The method of claim 1 wherein the midostaurin is administered orally.

12. The use of claim 7 wherein the midostaurin is administered orally.

13. The method of claim 2 wherein midostaurin is administered to the patient with the proviso that midostaurin is not to be used for simultaneous, separate or sequential use with imatinib.

14. The method of claim 3 wherein midostaurin is administered to the patient with the proviso that midostaurin is not to be used for simultaneous, separate or sequential use with imatinib.
